(19) United States
(12) Patent Application Publication
(10) Pub. No.: US 2011/0178384 A1
(43) Pub. Date: Jul. 21, 2011

(54) SYSTEM WITH AT LEAST ONE MEDICAL IMAGING DEVICE, AND METHOD TO PREPARE A SAMPLE FOR MEDICAL IMAGING

(76) Inventors: Rainer Kuth, Hoechstadt (DE); Sebastian Schmidt, Weisendorf (DE); Sabine Schaeffer-Kundler, Herzogenaurach (DE)

(21) Appl. No.: 13/011,189

(22) Filed: Jan. 21, 2011

(30) Foreign Application Priority Data
Jan. 21, 2010 (DE) 10 2010 001 092.8

(51) Int. Cl. A61B 5/05

(52) U.S. Cl. 600/407

(57) ABSTRACT

A system HAS at least one medical imaging device for implementing a medical imaging and at least one operating unit. The at least one operating unit is decoupled from the medical imaging device at least for a sample preparation for the medical imaging.

### BACKGROUND OF THE INVENTION

1. Field of the Invention

The present invention concerns a system with at least one medical imaging device for conducting a medical imaging procedure or session, and a method to prepare a sample for a medical imaging procedure or session.

2. Description of the Prior Art

Medical imaging devices are very expensive to purchase, so that, for cost recovery, as many examinations as possible should be conducted per day. However, in practice the greatest portion of time during which the medical imaging device is allocated for a patient is' used for preparation of the patient for the examination. For this purpose, the patient is positioned on a transport device of the medical imaging device. In addition, (for magnetic resonance imaging), accessories (for example local coils) are attached to the patient and measurement parameters required for the medical imaging, for example a slice position and/or a number of parameters, set within, or to form, a measurement workflow. Additionally, overview exposures of the patient may be produced or a planning of the slice position may be implemented. The actual examination or measurement involving medical imaging takes place only after this preparation phase, so the image data acquisition time (duration) of the occupies only a small part compared to the time of the preparation phase.

To save time, transport devices are known that can be decoupled from the medical imaging device. A portion of the patient preparation thus can be implemented separately from the medical imaging device. However, a coupling of the transport device to the medical imaging device is required for an adjustment of the measurement parameters.

Systems with a medical imaging device are known that additionally include an operating unit. The operating unit is permanently connected with the medical imaging device, so feedback from the medical imaging device is necessary to adjust (set) the measurement parameters.

### SUMMARY OF THE INVENTION

An object of the present invention is to provide a system with a medical imaging device in which a reservation time of the medical imaging device is significantly shortened to a measurement time for an examination measurement.

The invention is based on a system with at least one medical imaging device for medical imaging and at least one operating unit.

The at least one operating unit is decoupled from the medical imaging device for at least a sample preparation for the medical imaging. In this context, a medical imaging device means a device for tomographical imaging, for example a magnetic resonance tomography device or a computed tomography device. Furthermore, an operating unit means a unit that is designed for an adjustment of at least one measurement parameter, in particular using individual sample parameters and/or patient parameters, and/or for planning of slices that should be scanned in the examination measurement, and/or for generation of a workflow plan of an examination measurement, etc. For this purpose, the operating unit advantageously has at least one computer that can embody a processor unit and/or a memory unit so that general parameters and/or workflow programs can be created and/or stored. The operating unit also has an input unit for entry of individual (in particular sample-related) data by an operator of the operating unit. The operating unit can hereby be particularly inexpensively formed by a notebook, for example. A separate design and/or a separate and/or contact-free arrangement of the at least one operating unit apart from the at least one medical imaging device, means that neither a mechanical connection nor an electronic connection (for example a data transfer) between the operating unit and the medical imaging device is present. The functionality of the at least one operating unit for the sample preparation thus is fashioned essentially independently of the operation of the medical imaging device. A sample preparation means preparation of an examination subject, and particularly of a patient, for an examination measurement of the medical imaging device, so after the sample preparation the examination measurement can be started without additional adjustments being necessary.

With the embodiment according to the invention, the preparation of the sample (in particular of a patient) can ensue independently of the medical imaging device, and thus an allocation of the medical imaging device can be shortened, and in particular can be shortened essentially to the measurement time for the examination measurement of the medical imaging. The number of examination measurements per time unit can be advantageously increased, and therefore a high utilization (throughput) of the medical imaging can be achieved. For this purpose, measurement parameters and/or additional parameters are advantageously determined by the at least one operating unit for the sample preparation, and/or a workflow plan is established by the operating unit, etc., and these parameters are transmitted from the operating unit to the at least one medical imaging device, in particular before the examination measurement.

Furthermore, the system can have at least two operating units that are advantageously fashioned to be decoupled from the medical imaging device, at least for a sample preparation. An at least partially simultaneous sample preparation of different samples thus can be achieved. In addition, a sample preparation of a first sample and a simultaneous examination measurement of a second sample are also conceivable so that an effective utilization of the medical imaging device can be achieved. The system advantageously has a larger number of operating units than the number of medical imaging devices.

The at least one operating unit can be formed by a mobile operating unit, whereby a more flexible use of the operating unit can be achieved, for example for different medical imaging devices. In addition, preparation of patients that require a complicated preparation—for example preparation of infants—can take place in a familiar environment and/or at the hospital station, etc. by means of the mobile operating unit. In this context a mobile operating unit means an operating unit that can be moved and/or transported by an operator, separately and/or independently of the medical imaging device, and/or independently of the functionality of the medical imaging device.

The data transfer between the at least one operating unit and the medical imaging device can be achieved by the system having at least one interface unit for data exchange between the at least one operating unit and the medical imaging device. The interface unit can have an interface element for a direct coupling to the at least one operating unit at the medical imaging device, for example an interface element for connection of a data line; and/or an interface unit for a wireless data exchange. In addition the data transfer can be formed by means of external memory elements and/or transfer elements (for example a USB stick) that can be coupled with the desired system component via the interface unit.

In a further embodiment of the invention, the system has at least one transport device that can be coupled with the medical imaging device via a coupling unit. A transport device in this context means a device to transport a sample (in particular the patient) into or out of an acquisition region and/or an examination region of the medical imaging device. The sample (in particular the patient) is advantageously positioned on the transport device for preparation for the examination measurement so that the preparation for the examination measurement can subsequently begin. In the preparation phase the patient can already occupy an examination position on the transport device for the examination measurement. The coupling unit can be fashioned as a single (unitary) component with the interface unit for data transfer between the operating unit and the medical imaging device.

Furthermore, the system can have an additional interface unit that is designed for data exchange between the at least one operating unit and the transport device. An advantageous sample preparation thus can be achieved, for example by a position of the patient on the transport device and/or an examination region and/or anatomy of the patient being incorporated into a setting of the measurement parameters and/or into the workflow of a measurement program, etc. The additional interface unit can also include a mechanical coupling unit in addition to an interface unit for a data exchange, by means of which mechanical coupling unit the operating unit can be mechanically coupled to the transport device. Together with the transport device and/or via the transport device, the mobile operating unit can be connected and/or coupled with the medical imaging device.

A particularly space-saving arrangement of the operating unit can be achieved by fashioning the operating unit fashioned as one piece with the transport device. For example, a touch-sensitive surface for a data input for the operating unit can be integrated into the transport device. As used herein, “in one piece” means “formed by a module and/or by a unified structural unit”.

In a further embodiment of the invention, the system has an accessory unit that can be coupled with the at least one transport device by a coupling unit, so additional connections for a coupling to the medical imaging device can be avoided. Coupling and/or connection of the accessory unit with the medical imaging device advantageously ensues via the transport device, for example by the coupling of the transport device with the medical imaging device causing the corresponding control lines to be connected with one another. Cables and/or control lines advantageously run within the transport device so that an unwanted hindrance due to cables and/or conductors can be prevented. The accessory unit can, for example, be injectors for a contrast agent injection and/or an electrocardiogram unit (EKG unit) and/or local coils, etc. A parameterization and/or presetting of the accessory unit during the preparation phase of the patient and/or a control of the accessory unit by means of the operating unit can advantageously take place.

The at least one transport device can have at least one marking element to detect sample-specific information. The at least one marking element is advantageously fashioned to be touch-sensitive and/or pressure-sensitive at least in part, for example via a touch element and/or a pressure-sensitive sensor element. The transport device advantageously has multiple marking elements that are arranged on a bed surface of the transport device and/or are integrated into the bed surface, for example a touch-sensitive and/or pressure-sensitive mat that is integrated into the bed surface. The transport device can additionally have at least one marking element that is formed by a 3D scanner, for example a stereoscopic camera that takes different perspective exposures from which a three-dimensional image of the patient is subsequently determined. In this embodiment of the invention, the position of anatomical structures (for example a hip and/or a shoulder of the patient) and/or the weight of the patient and/or additional patient-related data can be detected by means of the transport device. In particular, an anatomical atlas of the patient can be produced and, using the atlas, slice planning and/or at least one measurement parameter and/or a workflow program can be adapted and/or created. The data detected by means of the marker elements are advantageously relayed to the operating unit and evaluated thereby.

In another embodiment of the invention, the system has at least two different medical imaging devices, and a selection of the respective medical imaging device for an examination measurement ensues via the at least one operating unit. A manual selection of the desired medical imaging can hereby be made by an operator via the operating unit and/or can take place automatically at least in part via the operating unit using selected parameters and/or using a selected examination area. For example, for a planned examination measurement a cardiac region of a patient, only those medical imaging devices of the system are selected by the operating unit, and/or are provided to the operator for selection that are equipped with an electrocardiogram unit and/or to which an electrocardiogram unit can be connected. In addition, a selection of the medical imaging device can ensue at least in part via the operating unit given the presence of a desired examination type, for example magnetic resonance tomography examination or computed tomography examination.

The at least one operating unit can have at least one simulation unit that simulates a medical imaging device for sample preparation that is selected in the operating unit. A virtual examination measurement can hereby take place using the set measurement parameters and/or additional parameters, and in particular the set measurement parameters and/or the additional parameters are optimized with regard to the virtual selected medical imaging device. For example, a contrast agent administration before the examination measurement can be adapted to a desired contrast in the shown virtual images, and/or a pulse sequence and/or a slice thickness can be adapted to a simulated and/or virtual contrast ratio. Contact of the mobile operating unit with the medical imaging device in the preparation phase of the sample can additionally be omitted, and the medical imaging device can be effectively used for examination measurements. Incorrect measurements due to incorrectly set measurement parameters can thus be reduced and/or prevented.

Furthermore, the invention encompasses a method for preparation of a sample for a medical imaging by means of a medical imaging device.

The preparation of the sample is at least partially implemented decoupled from the medical imaging device by means of the at least one operating unit decoupled from the medical imaging device. The preparation of the sample (a patient) can advantageously take place independent of the medical imaging device, and thus the usage time of the medical imaging device can advantageously be shortened, and in particular can be shortened to the measurement time for the actual examination measurement of the medical imaging. The number of examination measurements per time unit also can be increased, and therefore a high utilization of the medical imaging device can be achieved. For this measurement parameters and/or additional parameters are particularly advantageously determined and/or a workflow plan is established, etc. for the sample preparation by the at least one operating unit, and these parameters are transmitted from the operating unit to the at least one medical imaging device, such as before the examination measurement. In addition, the preparation of patients that require complicated preparation—for example preparation of infants—can take place in a familiar environment and/or at the station by means of the (in particular mobile) operating unit decoupled from the medical imaging device.

An advantageously short residence time in the imaging device by a patient can be achieved if at least one measurement parameter for the medical imaging is selected in a sample preparation phase via the at least one decoupled operating unit. An effective utilization in particular of different medical imaging devices and/or an effective adaptation and/or selection of measurement parameters and/or of additional parameters with regard to the medical imaging device can additionally be advantageously achieved.

Slice planning for the medical imaging can ensue via the at least one decoupled operating unit, so an advantageous time savings can be achieved given an allocation of the medical imaging device. An abstract atlas for a depiction of anatomy of the sample can be used for the slice planning of the sample. An atlas in this context means an abstract representation of anatomy of the sample (in particular the patient). Information and/or position markings of the anatomy of the patient and/or the weight of the patient also can be integrated into the atlas for this. The individual (in particular patient-related) position markings regarding the anatomy of the patient can be implemented by an input by an operator, for example by the operator communicating the position markings via at least one touch-sensitive and/or pressure-sensitive element so that a slice planning that is optimally adapted to a real anatomy of the patient can take place. Alternatively or additionally, the slice planning can be implemented using at least one exposure that is already present, this exposure already embodying individual (in particular patient-related) position markings.

An advantageous and time-saving transfer of the slice planning to the real (actual) anatomy of the sample and/or an advantageous adaptation of the slice planning to the anatomy of the sample—in particular of the patient—can be achieved if the atlas with the slice planning is calibrated with an overview exposure. The overview acquisition advantageously takes place after a data transfer of the at least one decoupled operating unit to the medical imaging device. The overview exposure ensues before the planned examination measurement and serves primarily to detect an exact anatomy of a target region and/or an examination region of the patient.

In a further embodiment, a virtual measurement by means of the decoupled operating unit is simulated for optimization of the measurement parameters. A virtual examination measurement can be implemented using the adjusted measurement parameters and/or additional parameters, and in particular the adjusted measurement parameters and/or the additional parameters can be matched to the selected medical imaging device. For example, administration of a contrast agent before the examination measurement can be adapted to a desired contrast in the presented virtual images, and/or a pulse sequence and/or a slice thickness can be adapted to a simulated and/or virtual contrast ratio.

### BRIEF DESCRIPTION OF THE DRAWINGS

FIG. 1 schematically illustrates a system according to the invention.

FIG. 2 shows an embodiment of the method according to the invention.

### DESCRIPTION OF THE PREFERRED EMBODIMENTS

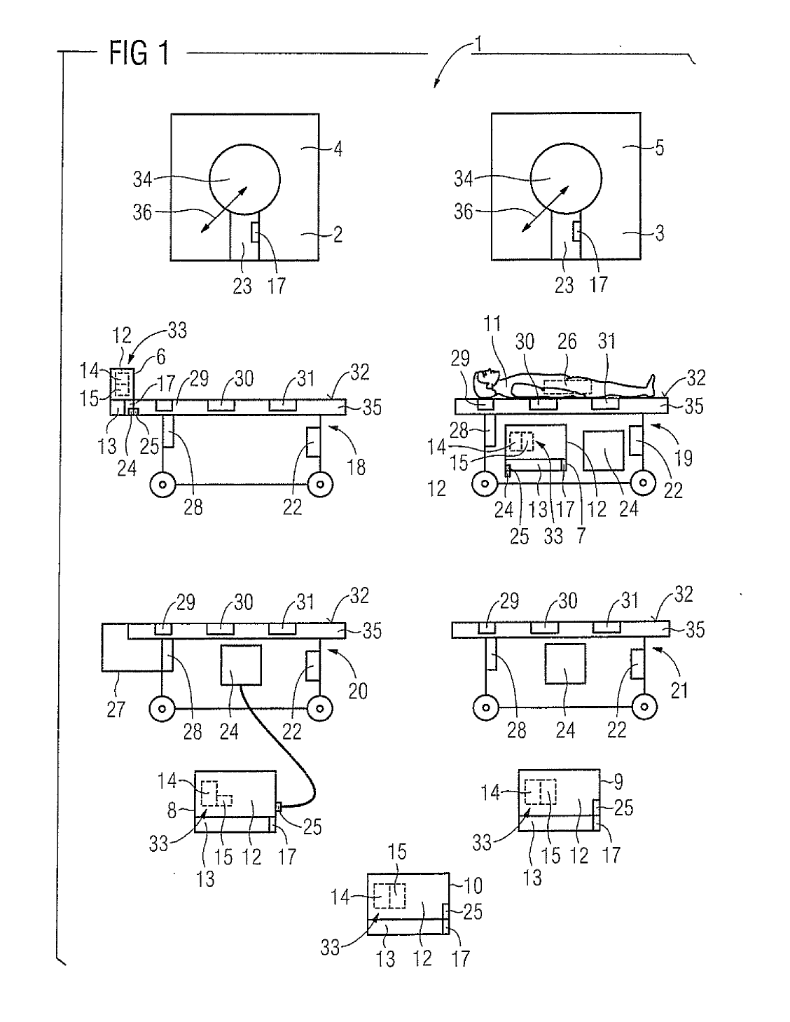

A system 1 with multiple medical imaging devices 2, 3 is shown in FIG. 1. The medical imaging devices 2, 3 are each a device for tomographical imaging and are in particular formed by two different magnetic resonance tomography devices. For example, the two magnetic resonance tomography devices 3, 4 can differ in magnetic field strength and/or in further parameters that appear to be reasonable to those skilled in the art. However, the number and/or a selection of the different medical imaging devices 2, 3 is not limited to the number and/or a selection of the medical imaging devices that are shown in FIG. 1. For example, the medical imaging devices 2, 3 could be computed tomography devices.

Furthermore, the system 1 has multiple operating units 6, 7, 8, 9, 10 that are separate from the respective medical imaging devices 2, 3 and that are decoupled from the medical imaging device 2, 3 for a sample preparation. The operation of the individual operating units 6, 7, 8, 9, 10 is thereby independent of the operation of the medical imaging device 2, 3 and/or of the distance and/or contact of the respective operating unit 6, 7, 8, 9, 10 from or with the medical imaging devices 2, 3. The operating units 6, 7, 8, 9, 10 are designed for a preparation of a sample (in particular of a patient 11) for a medical imaging examination by means of the medical imaging devices 2, 3, this preparation being separate or decoupled from the medical imaging devices 2, 3. For this purpose, the individual operating units 6, 7, 8, 9, 10 are respectively formed by mobile operating units 6, 7, 8, 9, 10 that can be moved and/or transported to a location of a patient preparation independently of the position of the medical imaging device. However, the number and/or embodiment of the different operating units 6, 7, 8, 9, 10 is not limited to the number and/or embodiment of the mobile operating units 6, 7, 8, 9, 10 shown in FIG. 1.

Each of the mobile operating units 6, 7, 8, 9, 10 has a monitor output 12 and an input unit 13 that is provided for a manual input of information and/or parameters. Furthermore, each of the mobile operating units 6, 7, 8, 9, 10 has a computer 14 that embodies a processor and a memory unit 15. The computer 14 additionally includes evaluation programs and/or control programs and/or additional computer programs that are stored in the memory unit 15. For data exchange between the individual mobile operating units 6, 7, 8, 9, 10 and the different medical imaging devices 2, 3, the mobile operating units 6, 7, 8, 9, 10 and the medical imaging units 2, 3 respectively have an interface unit 16, 17. The interface units 16, 17 are designed for wireless data exchange by means of a radio link—for example by means of Bluetooth and/or WLAN—between the operating units 6, 7, 8, 9, 10 and the medical imaging devices 2, 3. Furthermore, a data exchange by means of a data line is also possible.

The system 1 furthermore has multiple transport devices 18, 19, 20, 21. The transport devices 18, 19, 20, 21 are separate from the medical imaging devices 2, 3. Each of the transport devices 18, 19, 20, 21 can be coupled by means of a coupling unit 22 with one of the medical imaging devices 2, 3. However, the number and/or embodiment of the different transport devices 18, 19, 20, 21 is not limited to the number and/or embodiment of the transport devices 18, 19, 20, 21 shown in FIG. 1.

Each of the transport devices 18, 19, 20, 21 is formed at least in part from a magnetic resonance-compatible, non-magnetic and non-magnetizable material so that it can be used without interference for examination measurements 58 with the magnetic resonance tomography devices 4, 5. In addition, a design of the individual transport devices 18, 19, 20, 21 that is permeable to electromagnetic radiation (in particular x-ray radiation) is also conceivable, such that the transport devices 18, 19, 20, 21 can also be used for medical imaging devices 2, 3 that are formed by computed tomography devices. In addition to the transport devices 18, 19, 20, 21, the different medical imaging devices 2, 3 each has a coupling unit 23 corresponding to the coupling units 22 of the transport devices 18, 19, 20, 21, such that a coupling of the transport devices 18, 19, 20, 21 with each of the medical imaging devices 2, 3 is possible. The coupling units 22, 23 are provided for a mechanical coupling of the transport devices 18, 19, 20, 21 to one of the medical imaging devices 2, 3. The coupling units 22, 23 are also designed for data exchange between the transport devices 18, 19, 20, 21 and the medical imaging devices 2, 3. For this purpose, the coupling units 22, 23 can have a mechanical and/or electronic coupling means and/or additional coupling means that appear to be reasonable to those skilled in the art.

Furthermore, the system 1 includes additional interface units 24, 25 that are designed for a data exchange between the mobile operating units 6, 7, 8, 9, 10 and the transport devices 18, 19, 20, 21. The additional interface units 24, 25 can likewise be designed for wireless data exchange by means of a radio link (for example by means of Bluetooth and/or WLAN) as is shown between the mobile operating unit 9, 10 and the transport device 21 in FIG. 1. In addition to this, a data exchange by means of a data line is also possible as this is shown between the mobile operating units 8 and the transport device 20 in FIG. 1. The mobile operating unit 6, 7 and the transport device 18, 19 can also be fashioned as one piece with one another in that the mobile operating unit 6, 7 is integrated into the transport device 18, 19 (FIG. 1). For example, the operating unit 7 can be integrated into the transport device 19 as a touchscreen with associated computer 14.

The mobile operating units 6, 7, 8, 9, 10, together with the transport devices 18, 19, 20, 21, are designed for a preparation of the patient 11 for an imaging examination measurement 58 by means of the different medical imaging devices 2, 3. The patient preparation is implemented decoupled from the medical imaging device 2, 3 so that the medical imaging device 2, 3 is essentially occupied only for an examination measurement 58 of the patient 11. During the patient preparation the mobile operating unit 6, 7, 8, 9, 10 is thus without contact with the medical imaging device 2, 3 and is thus decoupled from the medical imaging devices 2, 3. The number of mobile operating units 6, 7, 8, 9, 10 and/or the number of transport devices 18, 19, 20, 21 is thereby greater than the number of medical imaging devices 2, 3 so that an additional patient can always be prepared for an examination measurement 58 during an examination measurement 58.

In a method to prepare the patient 11, in a first method step 50 the patient 11 is initially positioned on the transport device 18, 19, 20, 21 for the planned examination measurement 58. Due to the decoupled design of the transport device 18, 19, 20, 21 and the mobile design of the operating unit 6, 7, 8, 9, 10, this can take place outside of a treatment space that comprises the medical imaging device 2, 3, for example at a station and/or a room of the patient 11. In particular given patients 11 who require a complicated preparation (for example a preparation of infants), a long occupation time in the medical imaging device 2, 3 can hereby be prevented and the occupation time can be essentially reduced to an examination time.

In the first method step 50, additional hardware components of an accessory unit 27 of the system 1 can be additionally arranged and/or attached to the patient 11 depending on the planned examination measurement 58. For example, in the first method step 50 local coils are mounted around a planned examination region 26 of the patient 11 in which the planned examination measurement 58 should take place. Insofar as a contrast agent should be administered in the planned examination measurement 58, in a first method step 50 a contrast agent unit of the accessory unit 27 is connected to the transport device 20, for example. The accessory unit 27 can additionally contain further hardware components that are required for additional monitoring measurements in addition to the planned examination measurement 58 and/or monitoring measurements in parallel with the planned examination measurements, for example an electrocardiogram unit that acquires monitoring measurements of a cardiac region of the patient 11 in parallel with the planned examination measurement 58 by means of the medical imaging device 2, 3. The transport device 18, 19, 20, 21 has a coupling unit 28 for a connection to the accessory unit 27. In addition to this, electronic connections and/or data connections of the accessory unit 27 to the transport device 18, 19, 20, 21 are connected via the coupling unit 28 so that a connection of the accessory unit 27 to the medical imaging device 2, 3 occurs via the transport device 18, 19, 20, 21. Alternatively or additionally, the accessory unit 17 can be connected to the medical imaging device 2, 3 independent of the transport device 18, 19, 20, 21. Control and/or presetting and/or a parameterization of the accessory unit 27 in the preparation phase of the sample can hereby take place by means of the mobile operating unit 8.

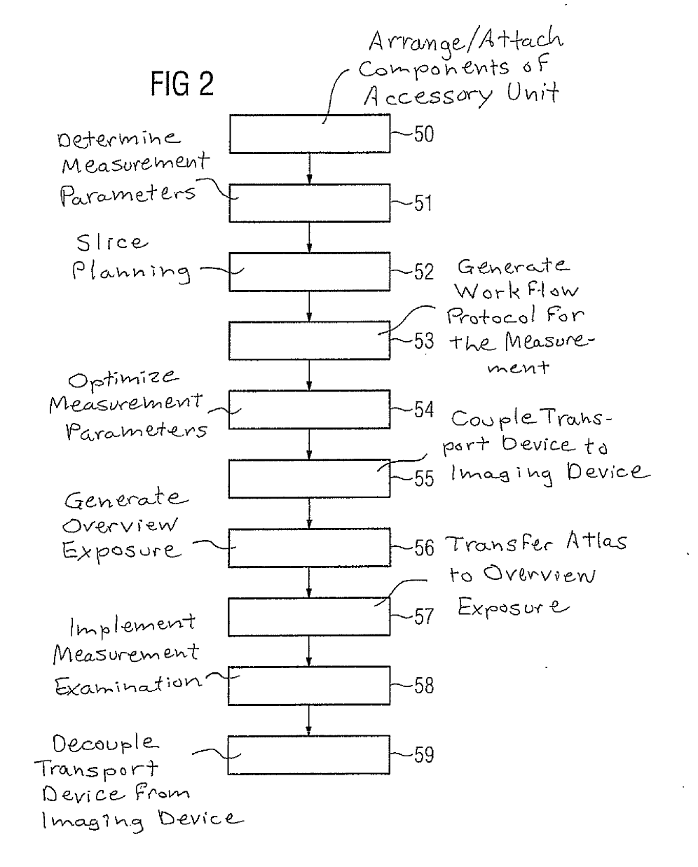

Measurement parameters for the imaging examination measurement 58 are subsequently determined in a measurement parameter determination method step 51 (FIG. 2). The determination of the measurement parameters is dependent on the medical imaging device 2, 3 that is selected for the examination measurement 58. For this purpose, the mobile operating unit 6, 7, 8, 9, 10 has a database with technical data of the different selectable medical imaging devices 2, 3 that, for example, contains a capacity of a gradient system of the medical imaging device 2, 3. The database is stored in the memory unit 15 of the mobile operating unit 6, 7, 8, 9, 10. An input and/or a selection of a measurement parameter is thereby limited by the selection of the medical imaging device 2, 3 within the mobile operating unit 6, 7, 8, 9, 10 in that the mobile operating unit 6, 7, 8, 9, 10 allows only inputs of the measurement parameters and/or limits the selection to measurement parameters that are matched to the selected medical imaging device 2, 3. Furthermore, entry of the measurement parameters for an abstract, virtual imaging device can take place by means of the mobile operating unit 6, 7, 8, 9, 10 and a conversion and/or adaptation of the input measurement parameters can subsequently take place—for example using a physical model in the computer 14 of the mobile operating unit 6, 7, 8, 9, 10—into measurement parameters for a medical imaging device 2, 3 that is provided for the examination measurement 58.

In addition to selection and/or limitation of the measurement parameters, a selection of the medical imaging device 2, 3 that is provided for the examination measurement 58 can take place via the mobile operating unit 6, 7, 8, 9, 10 using the input measurement parameters and/or additional information regarding the examination measurement 58. For example, given the selection of a cardiac examination by the operator of the mobile operating unit 6, 7, 8, 9, 10 a selection can be limited to medical imaging devices 2, 3 so that only medical imaging devices 2, 3 that are provided with an electrocardiogram unit (EKG unit) and/or to which the electrocardiogram unit can be connected are provided for the examination measurement 58. In a further embodiment of the invention it is also additionally conceivable that a selection of the medical imaging device 2, 3 takes place via a central computer of the system 1 that is connected with the individual mobile operating units 6, 7, 8, 9, 10 and/or medical imaging devices 2, 3, for example via Bluetooth and/or WLAN. For example, a selection of the medical imaging device 2, 3 could take place by utilization of the individual medical imaging devices 2, 3 of the system 1, for example by means of a planning system (RIS system) of the central computer, It is also possible for a selection to be made by the operator is transferred to the planning system.

After the input of the measurement parameters, a slice planning 52 takes place for the examination measurement 59. The slice planning 52 can take place using an atlas that shows an abstract representation and/or an abstract model of an anatomy of the patient 11. This atlas can additionally be adjusted and/or adapted with patient-related information to an actual anatomy of the patient 11. For this purpose, anatomical marking data are input by the operator and/or patient 11. The input of the anatomical marking data takes place via multiple marker elements 29, 30, 31 of the transport device 18, 19, 20, 21. The marker elements 29, 30, 31 are integrated into a bed surface 32 of a patient bed 35 of the transport device 18, 19, 20, 21. The marker elements 29, 30, 31 can be formed by touch-sensitive and/or pressure-sensitive sensor elements and/or by a touch-sensitive mat, and/or can be formed by additional marker elements that appear to be reasonable to the man skilled in the art. In particular, anatomical structures—for example a hip and/or a shoulder and/or a head area—are therefore registered via the marker elements 29, 30, 31. Furthermore, a weight of the patient 11 can be detected by means of the marker elements 29, 30, 31. The registration of the at least partial anatomical structure takes place via an explicit activation of the respective marker element 29, 30, 31 by the operator and/or patient 11, or particularly advantageously via an automatic triggering of the marker signal of the respective marker element 29, 30, 31, for example based on the weight of the patient 11 (who is positioned on the transport device 18, 19, 20, 21) that is acting on the marker element 29, 30, 31. The marking data are relayed to the operating unit 6, 7, 8, 9, 10 and there are integrated into the atlas.

Furthermore, at least one of the marker elements 29, 30, 31 can also be formed by a 3D scanner, for example by a stereoscopic camera that acquires different perspective exposures of the patient 11 and assembles these into a three-dimensional image so that optimally comprehensive information is obtained regarding the position and the anatomy of the patient 11. The slice planning 52 can additionally take place using already present anatomy exposures of the patient 11 that can be retrieved by the mobile operating unit 6, 7, 8, 9, 10 from a database. This database can be stored on the mobile operating unit 6, 7, 8, 9, 10 and/or be retrieved via a data line from a central database.

After an insertion of the patient-related information into the atlas via the mobile operating unit 6, 7, 8, 9, 10 and/or after an opening of the already present anatomy exposure of the patient 11, the actual slice planning 52 takes place in that the slices are plotted by an operator of the system (for example a physician) in the atlas and/or in the anatomy exposure. An input and/or the plotting of the slices hereby ensues by means of the input unit 13 of the mobile operating unit 6, 7, 8, 9, 10.

After the slice planning 52 for the examination measurement 58, a creation of a workflow protocol initially ensues in step 53 for the examination measurement 58. The individual measurement steps proceeding in series for the planned examination measurement 58 are established in the workflow protocol. The workflow protocol is automatically established by the computer 14 of the mobile operating unit 6, 7, 8, 9, 10 using the set and/or selected measurement parameters and/or the selected slices.

An optimization 54 of the measurement parameters by the mobile operating unit 6, 7, 8, 9, 10 takes place following the creation of the workflow protocol in step 53. The optimization 54 of the measurement parameters takes place decoupled from the medical imaging device 2, 3. For this the mobile operating unit 6, 7, 8, 9, 10 has a simulation unit 33 that simulates a virtual measurement using the workflow protocol and/or the selected measurement parameters for the selected medical imaging device 2, 3. The simulation unit 33 for this purpose has the computer 14 of the mobile operating unit 6, 7, 8, 9, 10 and a simulation software so that the simulation can be implemented by means of the computer 14 and the simulation software. In addition to patient-related data from the measurement parameters and/or from the workflow protocol, information and/or measurement parameters regarding the individual medical imaging devices 2, 3 formed by the system 1 enter into the simulation. The virtual, simulated measurement thus comprises optimally exact, real conditions of the medical imaging device 2, 3 and/or of the patient 11 and/or real measurement conditions so that the examination measurement 58 can be planned efficiently, decoupled from the medical imaging device 2. Using results of the virtual, simulated measurement, an optimization 54 of measurement parameters and/or additional adjustments of the planned examination measurement 58 then takes place. For example, a contrast agent administration before the examination measurement 58 can hereby be adapted to a desired contrast in the presented virtual images and/or a pulse sequence and/or a slice thickness can be adapted to a simulated and/or virtual contrast ratio. The results of the optimization 54 are presented to the operator via the screen output 12 of the mobile operating unit 6, 7, 8, 9, 10.

After the optimization 54 of the measurement parameters, the examination measurement 58 is completely planned and the patient 11 is transported by means of the transport device 18, 19, 20, 21 to the selected medical imaging device 2, 3. There the transport device 18, 19, 20 couples with the selected medical imaging device 2, 3 in a coupling step 55 by means of the coupling units 22, 23. A coupling and/or a connection of the accessory unit 27 and the mobile operating unit 6, 7, 8 (which is connected with the transport device 18, 19, 20 via the additional interface unit 24, 25) to the medical imaging device 2, 3 likewise takes place with the coupling of the transport device 18, 19, 20, 21 to the medical imaging device 2, 3. The interface unit 16 for data transfer of the mobile operating unit 6, 7, 8, 9, 10 with the interface 17 of the medical imaging device 2, 3 is fashioned in one piece with the coupling unit 23. Control lines and/or cables are thereby connected with one another upon coupling of the transport device 18, 19, 20 to the medical imaging device 2, 3 by means of the coupling units 22, 23. An information transfer and/or a data exchange in which the workflow protocol and/or additional parameters and/or information are transferred from the mobile operating unit 6, 7, 8, 9, 10 to the medical imaging device 2, 3 can additionally take place—wirelessly via the interface 16, 17 (for example via Bluetooth and/or WLAN) and/or via a data line—between the mobile operating unit 6, 7, 8, 9, 10 and the medical imaging device 2, 3. In an alternative embodiment of the invention, the data transfer between the mobile operating unit 6, 7, 8, 9, 10 and the medical imaging device 2, 3 can also take place by means of external, mobile storage elements—for example a USB stick—and/or the data to be transmitted can be placed in a memory element of a common data network, and a key for an access to the relevant data can be transmitted via the external, mobile storage elements, for example.

By means of the transport device 18, 19, 20, 21, the patient 11 is additionally introduced into an acquisition region 34 of the medical imaging device 2, 3 and there is positioned for the examination measurement 58. For this the patient bed 35 of the transport device 18, 19, 20, 21 is arranged such that it can be moved into the acquisition region 34 along a z-direction 36. As soon as the patient 11 is positioned by means of the patient bed 35 in the acquisition region 34 for the planned examination measurement 58, an overview exposure initially ensues of a target region and/or the examination region 26 of the patient 11. The overview exposure 56 serves to register the anatomy of the patient 11 in the target region and/or the examination region 26. The atlas of the patient 11 with the plotted slices that is created in the slice planning 52 is transferred onto the overview exposure 56 in a transfer step 57. Insofar as it is necessary, the slice planning 52 of the atlas is adapted to the overview exposure 56 in the transfer step 57.

The examination measurement 58 subsequently takes place according to the workflow program created by the mobile operating unit 6, 7, 8, 9, 10. After the examination measurement 58, a decoupling 59 of the transport device 18, 19, 20, 21 from the medical imaging device 2, 3 takes place. Together with the transport device 18, 19, 20, 21, the accessory unit 27 and/or the mobile operating unit 6, 7, 8, 9, 10 is thus also decoupled from the medical imaging device 2, 3. The patient 11, together with the operating unit 6, 7, 8, 9, 10 and the transport device 18, 19, 20, 21, can therefore be transported out of the examination room again so that the medical imaging device 2, 3 is made available for an additional examination measurement of an additional patient after the examination measurement 58.

Although modifications and changes may be suggested by those skilled in the art, it is the intention of the inventors to embody within the patent warranted hereon all changes and modifications as reasonably and properly come within the scope of their contribution to the art.

We claim as our invention:

1. A medical imaging system comprising:
at least one medical imaging device configured to interact with a subject to acquire image data from the subject, said subject requiring preparation prior to acquisition of said medical image data;
at least one computerized operating unit in communication, via a communication link, with said at least one medical imaging device, said computerized operating unit being configured to operate said medical imaging device to acquire said medical image data; and
said at least one computerized operating unit being configured to decouple said communication link with said at least one medical imaging device at least during said preparation of said subject.

2. A system as claimed in claim 1 comprising at least two computerized operating units each in communication with said at least one medical imaging device via respective communication links.

3. A system as claimed in claim 1 wherein said at least one computerized operating unit is a mobile computerized operating unit.

4. A system as claimed in claim 1 wherein said communication link comprises at least one interface unit for data exchange between said at least one computerized operating unit and said at least one medical imaging device.

5. A system as claimed in claim 4 wherein said interface unit is configured for wireless data exchange between said at least one computerized operating unit and said at least one medical imaging device.

6. A system as claimed in claim 1 comprising at least one transport device configured to transport the subject relative to said at least one medical imaging device, and a coupling unit that mechanically couples said at least one transport device with said at least one medical imaging device.

7. A system as claimed in claim 6 wherein said transport device is configured to receive signals from said at least one computerized operating unit via said communication link, and wherein said communication link comprises an interface unit for data exchange between said at least one computerized operating unit and said transport device.

8. A system as claimed in claim 6 wherein said at least one computerized operating unit and said transport device are formed as a unitary structural component.

9. A system as claimed in claim 6 comprising an accessory unit and a further coupling unit that mechanically couples said accessory unit with said transport device.

10. A system as claimed in claim 9 wherein said accessory unit is configured to receive signals from said at least one computerized operating unit for control or presetting of said accessory unit.

11. A system as claimed in claim 6 wherein said transport device comprises a marker element for registering information that is specific to said subject.

12. A system as claimed in claim 1 comprising two different medical imaging devices each in communication with said at least one computerized operating unit via respective communication links, and wherein said at least one computerized operating unit is configured to select one of said at least two different medical imaging devices for implementing acquisition of said image data from said subject.

13. A system as claimed in claim 12 wherein said at least one computerized operating unit comprises a computerized simulation unit that is configured to simulate said one of said two different medical imaging devices that has been selected, for preparation of said subject.

14. A system as claimed in claim 12 wherein at least one of said two different medical imaging devices is a magnetic resonance tomography device.

15. A method for preparation of a subject for medical imaging, comprising the steps of:
providing a medical imaging device that is in communication, via a communication link, with a computerized operating unit;
prior to acquiring medical image data from a subject by operating said medical imaging device via said communication link with said computerized operating device, preparing the subject for the acquisition of said medical image data; and
at least during preparation of said subject, decoupling said communication link between said medical imaging device and said computerized operating unit.

16. A method as claimed in claim 15, comprising:
while said computerized operating unit is decoupled from said medical imaging device, selecting, in said computerized operating unit, at least one measurement parameter for implementing the acquisition of said medical image data from the subject.

17. A method as claimed in claim 15, comprising:
acquiring said medical image data from the subject in a plurality of slices of the subject; and
while said computerized operating unit is decoupled from said medical imaging device, implementing slice planning in the computerized operating unit for the subsequent acquisition of said medical imaging data.

18. A method as claimed in claim 17, comprising:
from said computerized operating unit, accessing an anatomical atlas that depicts anatomy of the subject, and using said anatomical atlas for said slice planning.

19. A method as claimed in claim 18 comprising acquiring an overview exposure of the subject and additionally using said overview exposure to implement said slice planning in said computerized operating unit.

20. A method as claimed in claim 15, comprising:
while said computerized operating unit is decoupled from said medical imaging device, simulating a virtual acquisition of said medical imaging data from the subject in said computerized operating unit and using said virtual simulation to optimize measurement parameters for subsequently operating said medical imaging device to acquire said medical image data from the subject.
